(19) United States
(12) Patent Application Publication
(10) Pub. No.: US 2024/0099879 A1
(43) Pub. Date: Mar. 28, 2024

(54) DEVICES AND METHODS FOR SAFELY AND EFFECTIVELY RAISING OR MAINTAINING CORE BODY TEMPERATURE

(71) Applicants: Board of Regents, The University of Texas System, Austin, TX (US); Mercury Biomed, LLC, Cleveland, OH (US)

(72) Inventors: Kenneth R. Diller, Elgin, TX (US); Sepideh Khoshnevis, Austin, TX (US); Brian Patrick, Houston, TX (US); Patrick Brown, Cleveland Heights, OH (US)

(21) Appl. No.: 18/472,362

(22) Filed: Sep. 22, 2023

Related U.S. Application Data
(60) Provisional application No. 63/409,506, filed on Sep. 23, 2022.

(51) Int. Cl. A61F 7/08; A61F 7/00

(52) U.S. Cl. CPC A61F 7/08; A61F 7/007; A61F 2007/0086

(57) ABSTRACT

A method for regulating a core body temperature of a living subject may include removably coupling a flexible heat exchange pad to a skin surface of the living subject via a flexible adhesive composite laminate, and applying heat via the flexible heat exchange pad to the skin surface and underlying tissue of the living subject while the flexible heat exchange pad is coupled to the skin surface via the flexible adhesive composite laminate such that the applied heat regulates the core body temperature of the living subject.

### CROSS-REFERENCE TO RELATED APPLICATIONS

This application claims priority to U.S. Provisional Patent Application No. 63/409,506, filed Sep. 23, 2022, which is incorporated herein by reference in its entirety.

### GOVERNMENT SUPPORT CLAUSE

This invention was made with government support under Grant no. R42 GM119871 awarded by the National Institutes of Health. The government has certain rights in the invention.

### FIELD OF THE DISCLOSURE

The present disclosure relates generally to techniques for body temperature regulation and more particularly to devices and methods for safely and effectively raising or maintaining core body temperature of a living subject.

### BACKGROUND OF THE DISCLOSURE

Therapeutic heating may be used in various instances for regulating core body temperature of a living subject (e.g., a human subject or an animal subject). For example, therapeutic heating often may be provided while a subject is under general anesthesia and a surgical procedure is being performed on the subject. Therapeutic heating requires an elevated temperature of heat applied to a skin surface of the subject to cause an energy flow into the underlying tissue. However, elevation of the surface temperature must be limited to ensure against a thermal burn. Various types of existing heaters, such as water-flow heaters or air-flow heaters, may be used to provide therapeutic heating for a subject.

To be effective for therapeutic heating, a heater generally needs to balance the requirements of applying an elevated temperature of heat to the skin surface to induce energy flow into the underlying tissue and maintaining temperature at the skin surface to avoid thermal burn. Additionally, an effective heater should maintain good interfacial contact with the skin surface to facilitate the highest possible transmission of energy generated within the heater to the underlying skin to provide the largest therapeutic benefit for the subject. In this manner, the heater should avoid poor contact areas at which heat can accumulate rather than being transmitted to the skin, causing local hot spots that could lead to burns.

Various types of existing heaters fail to provide good interfacial contact with the skin surface to which the heater is applied. As a result, the use of such heaters may provide ineffective and/or inefficient transmission of energy to the target tissue and may lead to burns caused by poor contact areas. Certain existing heaters for therapeutic heating often may be used with straps or other types of mechanical devices to facilitate contact between the heater and the underlying skin surface. However, such devices often may apply mechanical pressure on underlying soft tissue, which may restrict blood flow and cause shear loading that, combined with tissue temperature elevated for therapy, can lead to causation of thermally enhanced pressure ulcer formation.

A need therefore exists for improved devices and methods for safely and effectively raising or maintaining core body temperature of a living subject, which may overcome one or more of the above-mentioned problems associated with existing temperature regulation technology.

### SUMMARY OF THE DISCLOSURE

The present disclosure provides flexible heat exchange pads, flexible adhesive composite laminates, heat exchange assemblies, and related methods of using the same for regulating a core body temperature of a living subject.

In one aspect, a method for regulating a core body temperature of a living subject is provided. In one embodiment, the method may include removably coupling a flexible heat exchange pad to a skin surface of the living subject via a flexible adhesive composite laminate, and applying heat via the flexible heat exchange pad to the skin surface and underlying tissue of the living subject while the flexible heat exchange pad is coupled to the skin surface via the flexible adhesive composite laminate such that the applied heat regulates the core body temperature of the living subject.

In some embodiments, at least a portion of the skin surface may include glabrous tissue. In some embodiments, an entirety of the skin surface may include glabrous tissue. In some embodiments, the skin surface may include at least a portion of a hand of the living subject. In some embodiments, the at least a portion of the hand of the living subject may include at least a portion of a palm of the hand. In some embodiments, the at least a portion of the hand of the living subject may include an entirety of a palm of the hand. In some embodiments, the skin surface may include at least a portion of a foot of the living subject. In some embodiments, the at least a portion of the foot of the living subject may include at least a portion of a sole of the foot. In some embodiments, the at least a portion of the foot of the living subject may include an entirety of a sole of the foot. In some embodiments, the living subject may be a mammalian subject. In some embodiments, the living subject may be a human subject. In some embodiments, the living subject may be an animal subject.

In some embodiments, the flexible heat exchange pad may be coupled to the living subject solely by the flexible adhesive composite laminate. In some embodiments, the flexible heat exchange pad may not be coupled to the living subject by any mechanical device or component other than the flexible adhesive composite laminate. In some embodiments, after coupling the flexible heat exchange pad to the skin surface of the living subject via the flexible adhesive composite laminate, no mechanical pressure may be applied to the skin surface or the underlying tissue through the flexible heat exchange pad. In some embodiments, after coupling the flexible heat exchange pad to the skin surface of the living subject via the flexible adhesive composite laminate, no mechanical pressure may be applied to the skin surface or the underlying tissue. In some embodiments, after coupling the flexible heat exchange pad to the skin surface of the living subject via the flexible adhesive composite laminate, no mechanical pressure may be applied to the flexible heat exchange pad.

In some embodiments, removably coupling the flexible heat exchange pad to the skin surface of the living subject via the flexible adhesive composite laminate may include conforming the flexible adhesive composite laminate to a contour of the skin surface. In some embodiments, removably coupling the flexible heat exchange pad to the skin surface of the living subject via the flexible adhesive composite laminate may include conforming the flexible heat exchange pad to a contour of the skin surface. In some embodiments, removably coupling the flexible heat exchange pad to the skin surface of the living subject via the flexible adhesive composite laminate may include conforming the flexible adhesive composite laminate and the flexible heat exchange pad to a contour of the skin surface. In some embodiments, at least a majority of the flexible adhesive composite laminate may be disposed between the flexible heat exchange pad and the skin surface while the flexible heat exchange pad is coupled to the skin surface via the flexible adhesive composite laminate. In some embodiments, an entirety of the flexible adhesive composite laminate may be disposed between the flexible heat exchange pad and the skin surface while the flexible heat exchange pad is coupled to the skin surface via the flexible adhesive composite laminate.

In some embodiments, the living subject may be under general anesthesia. In some embodiments, the flexible heat exchange pad may be coupled to the skin surface of the living subject via the flexible adhesive composite laminate before the living subject is placed under general anesthesia. In some embodiments, the flexible heat exchange pad may be coupled to the skin surface of the living subject via the flexible adhesive composite laminate after the living subject is placed under general anesthesia. In some embodiments, the flexible heat exchange pad may be coupled to the skin surface of the living subject via the flexible adhesive composite laminate before a surgical procedure is performed on the living subject. In some embodiments, the heat may be applied via the flexible heat exchange pad to the skin surface and underlying tissue of the living subject while a surgical procedure is performed on the living subject. In some embodiments, the applied heat may maintain the living subject in a normothermic state. In some embodiments, the applied heat may cause the living subject to transition from a hypothermic state to a normothermic state.

In some embodiments, the flexible adhesive composite laminate may include a flexible substrate having a first surface and a second surface disposed opposite one another, a first adhesive disposed on the first surface of the flexible substrate and configured for removably adhering to the skin surface, and a second adhesive disposed on the second surface of the flexible substrate and configured for removably adhering to the flexible heat exchange pad, with the second adhesive being different from the first adhesive. In some embodiments, the first adhesive may cover at least a majority of the first surface of the flexible substrate, and the second adhesive may cover at least a majority of the second surface of the flexible substrate. In some embodiments, the first adhesive may cover an entirety of the first surface of the flexible substrate, and the second adhesive may cover an entirety of the second surface of the flexible substrate. In some embodiments, the flexible substrate may include a film. In some embodiments, the flexible substrate may include a polymer. In some embodiments, the flexible substrate may include polyester. In some embodiments, the first adhesive may be biocompatible. In some embodiments, the second adhesive may be biocompatible. In some embodiments, the second adhesive may be non-biocompatible. In some embodiments, the first adhesive may be a pressure sensitive adhesive. In some embodiments, the first adhesive may be an acrylic adhesive. In some embodiments, the first adhesive may include rubber. In some embodiments, the second adhesive may be a high tack adhesive.

In some embodiments, the flexible heat exchange pad may have a first surface and a second surface disposed opposite one another, with the first surface of the flexible heat exchange pad facing toward the skin surface when the flexible heat exchange pad is coupled to the skin surface via the flexible adhesive composite laminate, and with the second surface of the flexible heat exchange pad facing away from the skin surface when the flexible heat exchange pad is coupled to the skin surface via the flexible adhesive composite laminate. In some embodiments, the flexible heat exchange pad may include a flexible heater defining the first surface of the flexible heat exchange pad and configured for generating heat, and a flexible thermally-insulative layer defining the second surface of the flexible heat exchange pad and configured for inhibiting heat transfer from the second surface of the flexible heat exchange pad. In some embodiments, the flexible heater may have constitutive properties such that the flexible heater is self-regulating against heating above a predetermined temperature. In some embodiments, the predetermined temperature may be less than or equal to 43° C. In some embodiments, the flexible heater may be a positive temperature coefficient heater. In some embodiments, the flexible heater may include a bus bar extending along a periphery of the flexible heater, and a plurality of heating elements extending inward from the bus bar. In some embodiments, each of the heating elements may have a linear shape extending in a linear manner from the bus bar. In some embodiments, the heating elements may extend parallel to one another. In some embodiments, the heating elements may be arranged in an array. In some embodiments, the flexible thermally-insulative layer may include a polymer.

In some embodiments, the flexible heat exchange pad also may include a plurality of feedback control temperature sensors. In some embodiments, the feedback control temperature sensors may be disposed between the flexible heater and the flexible thermally-insulative layer. In some embodiments, the feedback control temperature sensors may be coupled to a sensor strip. In some embodiments, each of the feedback control temperature sensors may include a resistance temperature detector. In some embodiments, the method also may include determining, via the feedback control temperature sensors, a plurality of temperature values, and controlling, via an electronic controller, the heat applied via the flexible heat exchange pad to the skin surface and the underlying tissue of the living subject based at least in part on the plurality of temperature values. In some embodiments, determining, via the feedback control temperature sensors, the plurality of temperature values may include determining a greater temperature value of the first temperature value and the second temperature value, comparing the greater temperature value and a predetermined maximum temperature setting, and controlling, via the electronic controller, the heat applied via the flexible heat exchange pad to the skin surface and the underlying tissue of the living subject based at least in part on the comparison of the greater temperature value and the predetermined maximum temperature setting. In some embodiments, controlling, via the electronic controller, the heat applied via the flexible heat exchange pad to the skin surface and the underlying tissue of the living subject based at least in part on the plurality of temperature values may include determining a differential between the first temperature value and the second temperature value, comparing the differential and a predetermined threshold limit, and controlling, via the electronic controller, the heat applied via the flexible heat exchange pad to the skin surface and the underlying tissue of the living subject based at least in part on the comparison of the differential and the predetermined threshold limit.

In another aspect, a flexible heat exchange pad for regulating a core body temperature of a living subject is provided. In one embodiment, the flexible heat exchange pad may include a flexible heater and a flexible thermally-insulative layer. The flexible heater may define a first surface of the flexible heat exchange pad and may be configured for generating heat. The first surface of the flexible heat exchange pad may be configured for facing toward a skin surface of the living patient when the flexible heat exchange pad is coupled to the skin surface. The flexible heater may have constitutive properties such that the flexible heater is self-regulating against heating above a predetermined temperature. The flexible thermally-insulative layer may define an opposite second surface of the flexible heat exchange pad and may be configured for inhibiting heat transfer from the second surface of the flexible heat exchange pad. The second surface of the flexible heat exchange pad may be configured for facing away from the skin surface when the flexible heat exchange pad is coupled to the skin surface.

In some embodiments, the flexible heater may be configured for conforming to a contour of the skin surface. In some embodiments, the predetermined temperature may be less than or equal to 43° C. In some embodiments, the flexible heater may be a positive temperature coefficient heater. In some embodiments, the flexible heater may include a bus bar extending along a periphery of the flexible heater, and a plurality of heating elements extending inward from the bus bar. In some embodiments, each of the heating elements may have a linear shape extending in a linear manner from the bus bar. In some embodiments, the heating elements may extend parallel to one another. In some embodiments, the heating elements may be arranged in an array. In some embodiments, the flexible thermally-insulative layer may include a polymer. In some embodiments, the flexible heat exchange pad also may include a plurality of feedback control temperature sensors. In some embodiments, the feedback control temperature sensors may be disposed between the flexible heater and the flexible thermally-insulative layer. In some embodiments, the feedback control temperature sensors may be coupled to a sensor strip. In some embodiments, the sensor strip may be disposed between the flexible heater and the flexible thermally-insulative layer. In some embodiments, each of the feedback control temperature sensors may include a resistance temperature detector. In some embodiments, the flexible heat exchange pad may be reusable.

In still another aspect, a flexible adhesive composite laminate for removably coupling a flexible heat exchange pad to a skin surface of a living subject for regulating a core body temperature of the living subject is provided. In one embodiment, the flexible adhesive composite laminate may include a flexible substrate, a first adhesive, and a second adhesive. The flexible substrate may have a first surface and a second surface disposed opposite one another. The first adhesive may be disposed on the first surface of the flexible substrate and configured for removably adhering to the skin surface. The second adhesive may be disposed on the second surface of the flexible substrate and configured for removably adhering to the flexible heat exchange pad, and the second adhesive may be different from the first adhesive.

In some embodiments, the first adhesive may cover at least a majority of the first surface of the flexible substrate, and the second adhesive may cover at least a majority of the second surface of the flexible substrate. In some embodiments, the first adhesive may cover an entirety of the first surface of the flexible substrate, and the second adhesive may cover an entirety of the second surface of the flexible substrate. In some embodiments, the flexible substrate may include a film. In some embodiments, the flexible substrate may include a polymer. In some embodiments, the flexible substrate may include polyester. In some embodiments, the first adhesive may be biocompatible. In some embodiments, the second adhesive may be biocompatible. In some embodiments, the second adhesive may be non-biocompatible. In some embodiments, the first adhesive may be a pressure sensitive adhesive. In some embodiments, the first adhesive may be an acrylic adhesive. In some embodiments, the first adhesive may include rubber. In some embodiments, the second adhesive may be a high tack adhesive. In some embodiments, the flexible adhesive composite laminate also may include a first cover removably covering the first adhesive and the first surface of the flexible substrate and configured for being removed therefrom prior to adhering the first adhesive to the skin surface, and a second cover removably covering the second adhesive and the second surface of the flexible substrate and configured for being removed therefrom prior to adhering the second adhesive to the flexible heat exchange pad. In some embodiments, the flexible adhesive composite laminate may be disposable.

In yet another aspect, a heat exchange assembly for regulating a core body temperature of a living subject is provided. In one embodiment, the heat exchange assembly may include a flexible heat exchange pad and a flexible adhesive composite laminate. The flexible heat exchange pad may include a flexible heater and a flexible thermally-insulative layer. The flexible heater may define a first surface of the flexible heat exchange pad and may be configured for generating heat. The first surface of the flexible heat exchange pad may be configured for facing toward a skin surface of the living patient when the flexible heat exchange pad is coupled to the skin surface. The flexible heater may have constitutive properties such that the flexible heater is self-regulating against heating above a predetermined temperature. The flexible thermally-insulative layer may defining an opposite second surface of the flexible heat exchange pad and may be configured for inhibiting heat transfer from the second surface of the flexible heat exchange pad. The second surface of the flexible heat exchange pad may be configured for facing away from when the flexible heat exchange pad is coupled to the skin surface. The flexible adhesive composite laminate may include a flexible substrate, a first adhesive, and a second adhesive. The flexible substrate may have a first surface and a second surface disposed opposite one another. The first adhesive may be disposed on the first surface of the flexible substrate and configured for removably adhering to the skin surface. The second adhesive may be disposed on the second surface of the flexible substrate and configured for removably adhering to the flexible heat exchange pad, and the second adhesive may be different from the first adhesive.

In some embodiments, the flexible heat exchange pad and the flexible thermally-insulative layer may be configured for conforming to a contour of the skin surface. In some embodiments, the predetermined temperature may be less than or equal to 43° C. In some embodiments, the flexible heater may be a positive temperature coefficient heater. In some embodiments, the flexible heater may include a bus bar extending along a periphery of the flexible heater, and a plurality of heating elements extending inward from the bus bar. In some embodiments, each of the heating elements may have a linear shape extending in a linear manner from the bus bar. In some embodiments, the heating elements may extend parallel to one another. In some embodiments, the heating elements may be arranged in an array. In some embodiments, the flexible thermally-insulative layer may include a polymer. In some embodiments, the flexible heat exchange pad also may include a plurality of feedback control temperature sensors. In some embodiments, the feedback control temperature sensors may be disposed between the flexible heater and the flexible thermally-insulative layer. In some embodiments, the feedback control temperature sensors may be coupled to a sensor strip. In some embodiments, the sensor strip may be disposed between the flexible heater and the flexible thermally-insulative layer. In some embodiments, each of the feedback control temperature sensors may include a resistance temperature detector. In some embodiments, the flexible heat exchange pad may be reusable.

In some embodiments, the first adhesive may cover at least a majority of the first surface of the flexible substrate, and the second adhesive may cover at least a majority of the second surface of the flexible substrate. In some embodiments, the first adhesive may cover an entirety of the first surface of the flexible substrate, and the second adhesive may cover an entirety of the second surface of the flexible substrate. In some embodiments, the flexible substrate comprises a film. In some embodiments, the flexible substrate may include a polymer. In some embodiments, the flexible substrate may include polyester. In some embodiments, the first adhesive may be biocompatible. In some embodiments, the second adhesive may be biocompatible. In some embodiments, the second adhesive may be non-biocompatible. In some embodiments, the first adhesive may be a pressure sensitive adhesive. In some embodiments, the first adhesive may be an acrylic adhesive. In some embodiments, the first adhesive may include rubber. In some embodiments, the second adhesive may be a high tack adhesive. In some embodiments, the flexible adhesive composite laminate also may include a first cover removably covering the first adhesive and the first surface of the flexible substrate and configured for being removed therefrom prior to adhering the first adhesive to the skin surface, and a second cover removably covering the second adhesive and the second surface of the flexible substrate and configured for being removed therefrom prior to adhering the second adhesive to the flexible heat exchange pad. In some embodiments, the flexible adhesive composite laminate may be disposable. In some embodiments, the flexible heat exchange pad may have a first footprint, and the flexible adhesive composite laminate may have a second footprint that is equal to or less than the first footprint.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A is a perspective view of an example temperature regulation system for regulating a core body temperature of a living subject in accordance with one or more embodiments of the disclosure, showing a plurality of flexible heat exchange pads, a control module, a plurality of cables, and a support structure of the temperature regulation system.

FIG. 1B is a perspective view of one of the flexible heat exchange pads of the temperature regulation system of FIG. 1A, showing a flexible heater of the flexible heat exchange pad along an active surface thereof.

FIG. 1C is a perspective view of one of the flexible heat exchange pads of the temperature regulation system of FIG. 1A, showing a flexible thermally-insulative layer and an electronics module of the flexible heat exchange pad along an inactive surface thereof.

FIG. 1D is an exploded perspective view of one of the flexible heat exchange pads of the temperature regulation system of FIG. 1A, showing the flexible heater, the flexible thermally-insulative layer, the electronics module, and sensor strip with a plurality of feedback control temperature sensors of the flexible heat exchange pad.

FIG. 1E is a perspective view of one of the flexible heat exchange pads of the temperature regulation system of FIG. 1A, illustrating connection of one of the cables to the flexible heat exchange pad.

FIG. 1F is a perspective view of a plurality of flexible adhesive composite laminates of the temperature regulation system of FIG. 1A.

FIG. 1G is an exploded perspective view of one of the flexible adhesive composite laminates of the temperature regulation system of FIG. 1A, showing a flexible substrate, a first adhesive, a second adhesive, a first cover, and a second cover of the flexible adhesive composite laminate.

FIG. 1H is a perspective view of the control module of the temperature regulation system of FIG. 1A, showing the control module mounted to the support structure and having the cables coupled thereto.

FIG. 1I is a perspective view of the control module of the temperature regulation system of FIG. 1A.

FIG. 1J is a perspective view of the control module of the temperature regulation system of FIG. 1A, showing a user interacting with a user interface of the control module.

FIG. 1K is a perspective view of the control module of the temperature regulation system of FIG. 1A, showing a user interacting with the user interface of the control module.

FIG. 1L is a perspective view of the control module of the temperature regulation system of FIG. 1A.

FIG. 1M is an exploded perspective view of the control module of the temperature regulation system of FIG. 1A.

FIGS. 1N-1S are perspective views illustrating coupling of one of the flexible adhesive composite laminates to one of the flexible heat exchange pads of the temperature regulation system of FIG. 1A.

FIGS. 1T and 1U are perspective views illustrating coupling of one of the flexible heat exchange pads to a hand of a human subject via one of the flexible adhesive composite laminates of the temperature regulation system of FIG. 1A.

FIG. 1V is a perspective view illustrating coupling of one of the flexible heat exchange pads to a foot of a human subject via one of the flexible adhesive composite laminates of the temperature regulation system of FIG. 1A.

FIGS. 2A and 2B are perspective views illustrating an experimental test setup for studying the effect of the flexible adhesive composite laminate being used to couple the flexible heat exchange pad of the temperature regulation system of FIG. 1A to a hand of a human subject.

FIG. 2C is a graph of current input as a function of time, illustrating example data obtained with the subject's hand face down on the flexible heat exchange pad, without the flexible adhesive composite laminate being used.

FIG. 2D is a graph of power input as a function of time, illustrating example data obtained with the subject's hand face down on the flexible heat exchange pad, without the flexible adhesive composite laminate being used.

FIG. 2E is a graph of feedback temperature as a function of time, illustrating example data obtained with the subject's hand face down on the flexible heat exchange pad, without the flexible adhesive composite laminate being used.

FIG. 2F is a graph of hand and heater temperatures as a function of time, illustrating example data obtained with the subject's hand face down on the flexible heat exchange pad, with the flexible adhesive composite laminate being used.

FIG. 2G is a graph of current input as a function of time, illustrating example data obtained with the subject's hand face down on the flexible heat exchange pad, with the flexible adhesive composite laminate being used.

FIG. 2H is a graph of power input as a function of time, illustrating example data obtained with the subject's hand face down on the flexible heat exchange pad, with the flexible adhesive composite laminate being used.

FIG. 2I is a graph of feedback temperature as a function of time, illustrating example data obtained with the subject's hand face down on the flexible heat exchange pad, with the flexible adhesive composite laminate being used.

FIG. 2J is a graph of hand and heater temperatures as a function of time, illustrating example data obtained with the subject's hand face down on the flexible heat exchange pad, with the flexible adhesive composite laminate being used.

FIG. 2K is a graph of current input as a function of time, illustrating example data obtained with the subject's hand face up on the flexible heat exchange pad, without the flexible adhesive composite laminate being used.

FIG. 2L is a graph of power input as a function of time, illustrating example data obtained with the subject's hand face up on the flexible heat exchange pad, without the flexible adhesive composite laminate being used.

FIG. 2M is a graph of feedback temperature as a function of time, illustrating example data obtained with the subject's hand face up on the flexible heat exchange pad, without the flexible adhesive composite laminate being used.

FIG. 2N is a graph of hand and heater temperatures as a function of time, illustrating example data obtained with the subject's hand face up on the flexible heat exchange pad, with the flexible adhesive composite laminate being used.

FIG. 2O is a graph of current input as a function of time, illustrating example data obtained with the subject's hand face up on the flexible heat exchange pad, with the flexible adhesive composite laminate being used.

FIG. 2P is a graph of power input as a function of time, illustrating example data obtained with the subject's hand face up on the flexible heat exchange pad, with the flexible adhesive composite laminate being used.

FIG. 2Q is a graph of feedback temperature as a function of time, illustrating example data obtained with the subject's hand face up on the flexible heat exchange pad, with the flexible adhesive composite laminate being used.

FIG. 2R is a graph of hand and heater temperatures as a function of time, illustrating example data obtained with the subject's hand face up on the flexible heat exchange pad, with the flexible adhesive composite laminate being used.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, and components have not been described in detail so as not to unnecessarily obscure aspects of the embodiments.

Overview

Embodiments of temperature regulation systems, flexible heat exchange pads, flexible adhesive composite laminates, heat exchange assemblies, and related methods of using the same for regulating a core body temperature of a living subject are provided herein. As described herein, the disclosed devices and methods may overcome one or more of the above-mentioned problems associated with existing temperature regulation technology. For example, a flexible heat exchange pad may be removably coupled to a skin surface of a subject by a flexible adhesive composite laminate, without the need for straps or other mechanical devices. In this manner, the present technology may avoid application of mechanical pressure to underlying soft tissue during therapeutic heating. Accordingly, use of the flexible adhesive composite laminate may avoid restricting blood flow and causing shear loading that, combined with tissue temperature elevated for therapy, can lead to causation of thermally enhanced pressure ulcer formation. Additionally, by eliminating the need for straps or other mechanical devices, use of the flexible heat exchange pad coupled to the skin surface of the subject by the flexible adhesive composite laminate may be particularly advantageous at locations near where a peripherally inserted central catheter (PICC) line is inserted into the subject. For example, the flexible heat exchange pad may be coupled to the palm of the subject's hand by the flexible adhesive composite laminate, while the PICC line may be inserted along the back of the subject's hand or arm. In this manner, delivery of fluids to the subject via the PICC line may be uninhibited by the flexible heat exchange pad and the flexible adhesive composite laminate, in contrast to such fluid delivery being adversely affected by mechanical straps used with certain conventional heaters. Moreover, the flexibility of the heat exchange pad and the adhesive composite laminate may allow them to easily conform to the complex contours of skin surfaces of various areas of the body, including the palms of the hands and the soles of the feet. In this manner, use of the flexible heat exchange pad and the flexible adhesive composite laminate may provide optimal interfacial contact with the skin surface, allowing for highly effective and efficient transmission of energy to the underlying skin and soft tissue while also avoiding poor contact areas that can lead to thermal burn.

As described herein, use of the flexible heat exchange pad coupled to the palms of the hands and soles of the feet may be particularly beneficial, as these body portions constitute the primary areas of the skin containing heat transfer vasculature, arteriovenous anastomoses (AVAs), that serve as the highest efficiency convective heat transfer portals for the exchange of heat between flowing blood (to and from the body core) and the environment. These surfaces are key to the functioning of the human thermoregulatory system and offer the greatest opportunity for manipulating the core temperature during anesthetized surgery to avoid perioperative hypothermia with its attendant dangers for leading to bleeding disorders and enhanced susceptibility to infection. The flexible heat exchange pads, when coupled to the palms and the soles by the flexible adhesive composite laminates, are uniquely adapted to blocking heat loss from AVA blood flow during anesthesia medicated vasodilation and to adding or removing heat from AVA blood flow as the therapeutic need may dictate.

The flexible heat exchange pad may include a flexible heater configured for generating heat and a flexible thermally-insulative layer inhibiting heat transfer in a direction away from the skin surface. As described herein, the flexible heater may be a positive temperature coefficient (PTC) heater that includes a plurality of heating elements arranged in a tight geometric density and supplied with electrical energy via a peripheral low resistance bus bar to create a heating pattern that is far more uniform than what is produced by traditional embedded wire coil resistance heaters and flowing water channel heaters that are susceptible to developing local hot spots and substantial local temperature gradients. The constitutive properties of the PTC heater may be selected and manipulated so that the heater has a strong tendency toward being self-limiting against overheating above 43° C. to reduce the risk of burn injury causation. The inherent property of PTC heaters being self-regulating and self-limiting in their temperature output may be particularly useful to exploit to produce safe and effective thermal therapy applications. Although PTC heaters may be particularly well suited for the flexible heat exchange pad, other types of suitable heaters may be used in various embodiments. Most heating pads function based on PR energy dissipation in resistance conductors embedded in a support matrix. This method may use Joule heating as the fundamental mechanism. Both direct current and alternating current activation may be used in various embodiments. Significant variations in heater design may be achieved by the geometric pattern of the resistance conductors. The heating level typically may be regulated by (i) constitutive resistance of the conductor that is set during device design and (ii) power applied to the heater via in-process modulation of the applied voltage and current. Limitation of upper temperature levels rely on setting the inherent resistance of the electrical conductor in combination with regulation of applied voltage. This generally provides imprecise control and limits the ability to regulate the risk of injury. Alternatively, a heater may be equipped with temperature readout instrumentation that can provide signals for feedback control. Traditional electrical resistance heaters have no inherent limitation on how high the temperature can go as the input power (voltage and current) are progressively increased. Various means of electric power other than Joule heating can be used for driving an electric heater. One example is induction coupling, according to which an alternating electrical current is applied to activate an induction coil that produces an alternating magnetic field that causes both hysteresis loses in a magnetic material and eddy loses in any conductive material, which loses result in internal heating. The object to be heated has to be placed internally to an induction coil, which is not practical for the current application. Many other heating mechanisms exist. Virtually all processes have irreversibilities that result in internal energy dissipation to produce heating. However, very few of these effects can be applied to warm the surface of tissues because the internal heating needs to be regulated and applied geometrically to the intended target. As described herein, the present technology provides unique attributes that allow it to be applied to the skin surface with controlled efficacy and with very limited risk of causing thermal heating injury.

As described herein, the present technology may implement a feedback control system that operates based on input from a plurality of temperature sensors, such as two temperature sensors small resistance temperature detectors (RTDs), mounted between the flexible heater and the flexible thermally-insulative layer, on the surface of the flexible heater opposite that which faces the skin surface during therapeutic heating. The control system may use the highest of the two temperatures detected by the RTDs as the regulating input to the heater. In some instances, if the differential between the two temperature values detected by the respective sensors exceeds a threshold limit for a defined period of time, the system may be deemed to be in a default condition and may be immediately shut down. In some instances, a maximum temperature of the controller may be set to a particular temperature value, such as 41° C., that provides a margin of safety below the threshold of 43° C. above which thermal burn injury can occur. The combination of dual sensor feedback control and PTC constitutive behavior of the heater may provide a higher level of safety against burn injury causation than either control mechanism independently can provide, which is not available on existing systems for heating the skin surface.

The flexible thermally-insulative layer of the flexible heat exchange pad may facilitate multiple performance and safety benefits. For example, the flexible thermally-insulative layer may provide thermal isolation from the environment, enabling more accurate control of the heater temperature and thus safer operation. Additionally, the thermal barrier effect produced by the flexible thermally-insulative layer may ensure that the hottest region of the heater is where the feedback control temperature sensors are located, ensuring safety on the active surface of the heater which applies therapeutic heating to the skin. Moreover, the thermal isolation of the heater means that energy generated therein advantageously may be directed toward the active surface contacting the skin for therapeutic benefit, conserving resources that are especially important when the system is used with battery power during transport of a patient. The flexible thermally-insulative layer also ensures that healthcare personnel may not touch a hot area of the heater.

In some instances, the heater of the flexible heat exchange pad and the control system of the temperature regulation system may be selectively operated with either battery power (DC) or mains (AC) electrical power. This ability to selectively switch between power sources may enable both long term stationary use as well as mobile use during patient transport, with switching between DC and AC power modes allowing for continuous, uninterrupted operation of the temperature regulation system.

As described herein, the flexible adhesive composite laminate may include a flexible substrate with adhesive material disposed on opposite sides of the substrate. A first adhesive disposed on one side of the flexible substrate may be used for adhering to the skin surface, while a second adhesive disposed on the opposite side of the flexible substrate may be used for adhering to the active surface of the flexible heat exchange pad. Various suitable materials may be used for the flexible substrate and the adhesives. In some instances, the flexible substrate may be formed of, or may include, a polymer, such as polyester, and may be formed as a film. In some instances, the first adhesive may be a biocompatible adhesive, while the second adhesive may be a non-biocompatible adhesive. According to one example, the substrate may be 3M Scotchpak 9758 polyester film, the first adhesive may be biocompatible 3M 1587 rubber-based PSA, and the second adhesive may be 3M 9425HT high tack adhesive.

The flexible adhesive composite laminate may provide multiple major safety and functional advantages for attaching the heater to the skin as compared to traditional methods, such as mechanical straps. The adhesive composite laminate may provide a continuous contact area over the entire therapeutic target surface, such as the palm, fingers, sole, and toes. The continuous contact may increase the surface area for heat transfer, thereby enabling a greater net heat flow to the therapeutic area. Additionally, the continuous contact may produce a more uniform temperature pattern on the skin surface, resulting in more effective boundary conditions for heat transfer to the skin. The adhesive composite laminate advantageously may obviate the need to use mechanical means to hold the heater to the skin surface, eliminating local stress concentrations in the soft tissue areas underlying the straps. These stress concentrations may be dangerous artifacts when applied to soft tissue to hold a heat exchange pad in place because the surface stresses are transmitted into the underlying soft tissue, resulting in matching patterns of localized ischemia and cell shearing that issue directly in the formation of pressure ulcers. Eliminating this safety risk associated with traditional strap-mounted heaters is a significant advantage of the present technology.

The disclosed temperature regulation systems, flexible heat exchange pads, flexible adhesive composite laminates, heat exchange assemblies, and related methods of using the same advantageously may solve the challenge of producing a large heat flux (W/cm2) into the skin surface without raising the temperature so high as to cause burns and without applying mechanical compression to a body part in order to hold a heater tightly to the skin surface. As discussed above, such compression may restrict blood flow in underlying soft tissue and induce shear stress on cells that collectively lead to pressure ulcer formation that may be exacerbated by elevated therapeutic temperatures. Moreover, the flexibility of the heat exchange pads and the flexible adhesive composite laminates enable them to conform to complex three-dimensional anatomical shapes, especially as are encountered on the palms and the soles.

Still other benefits and advantages of the temperature regulation systems, flexible heat exchange pads, flexible adhesive composite laminates, heat exchange assemblies, and related methods provided herein over existing temperature regulation technology will be appreciated by those of ordinary skill in the art from the following description and the appended drawings.

Example Temperature Regulation Systems, Flexible Heat Exchange Pads, Flexible Adhesive Composite Laminates, Heat Exchange Assemblies, and Related Methods

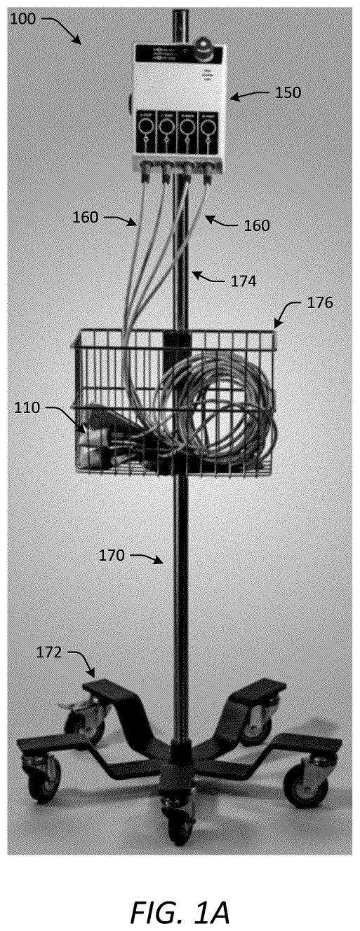

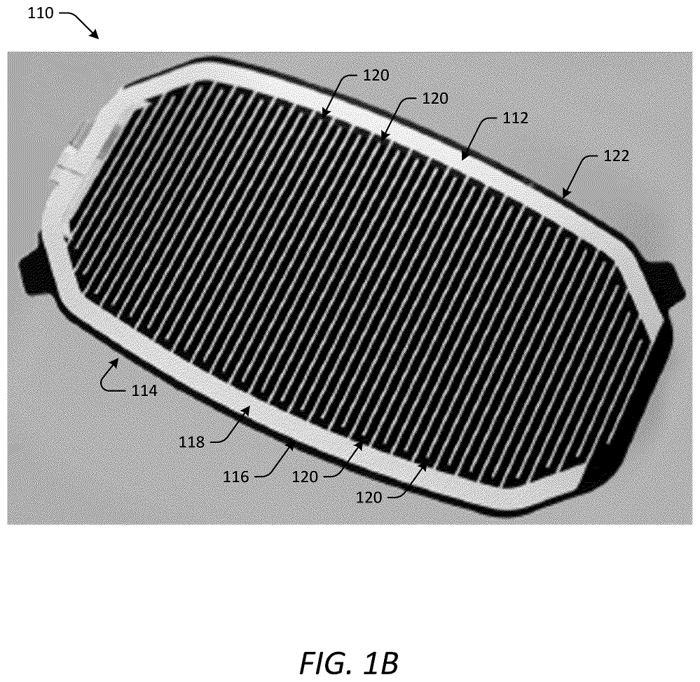

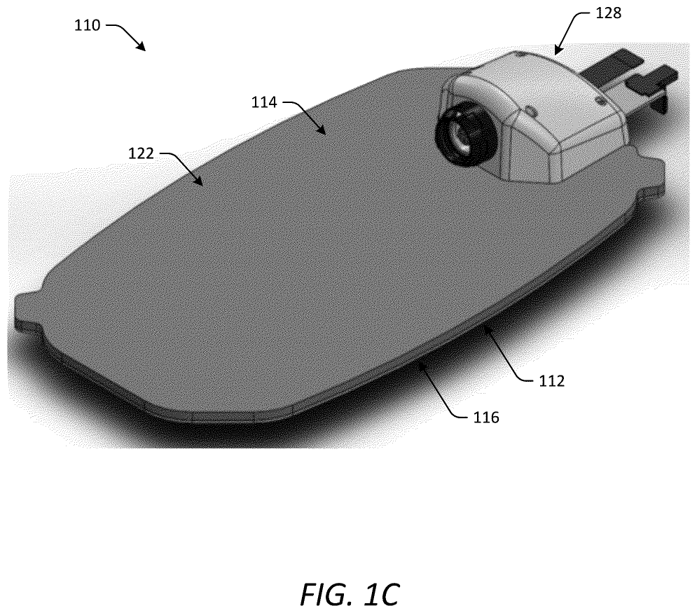

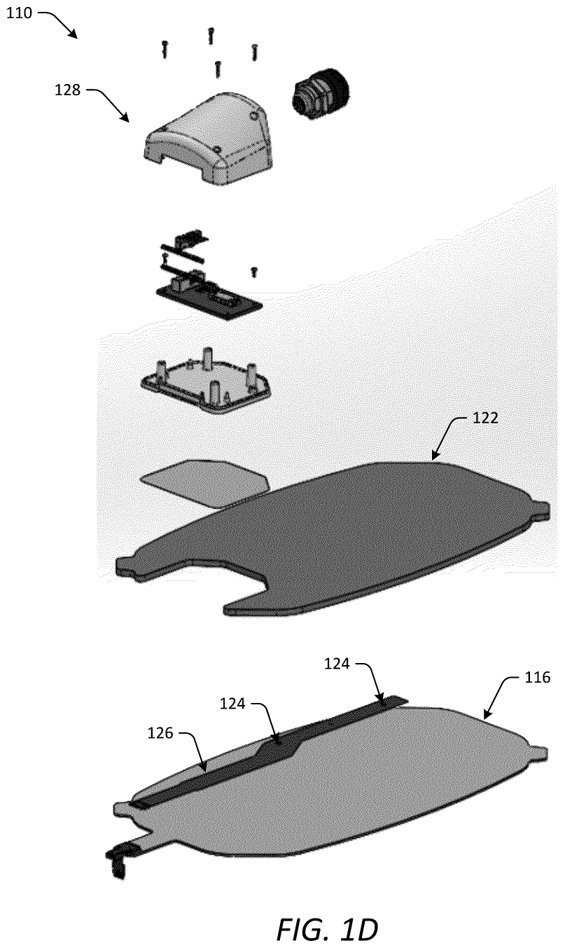

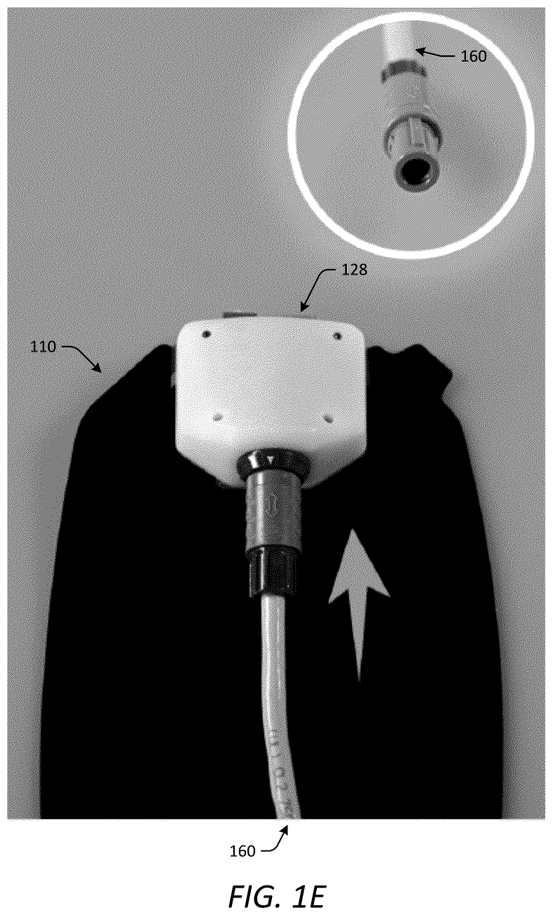

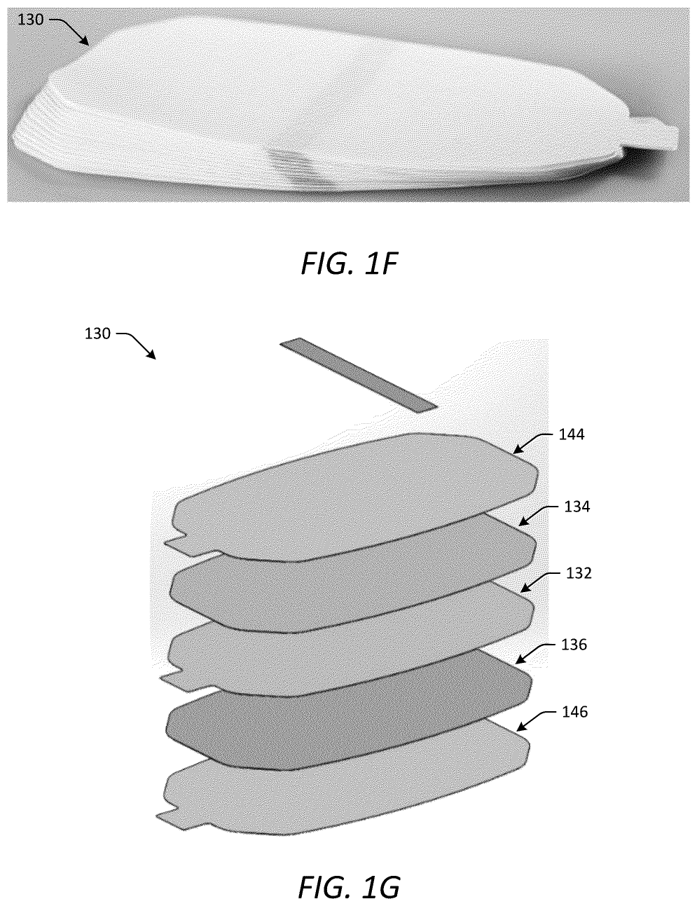

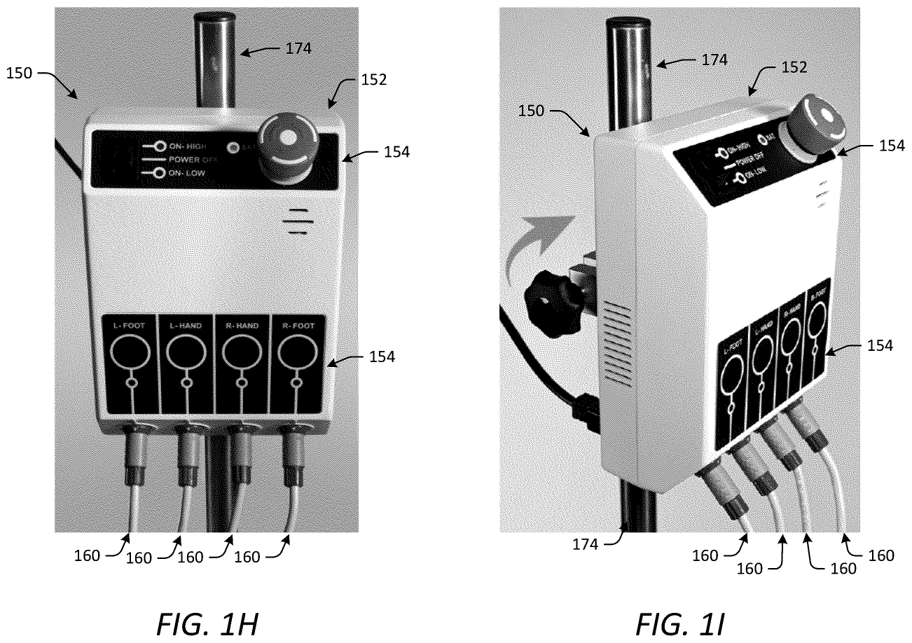

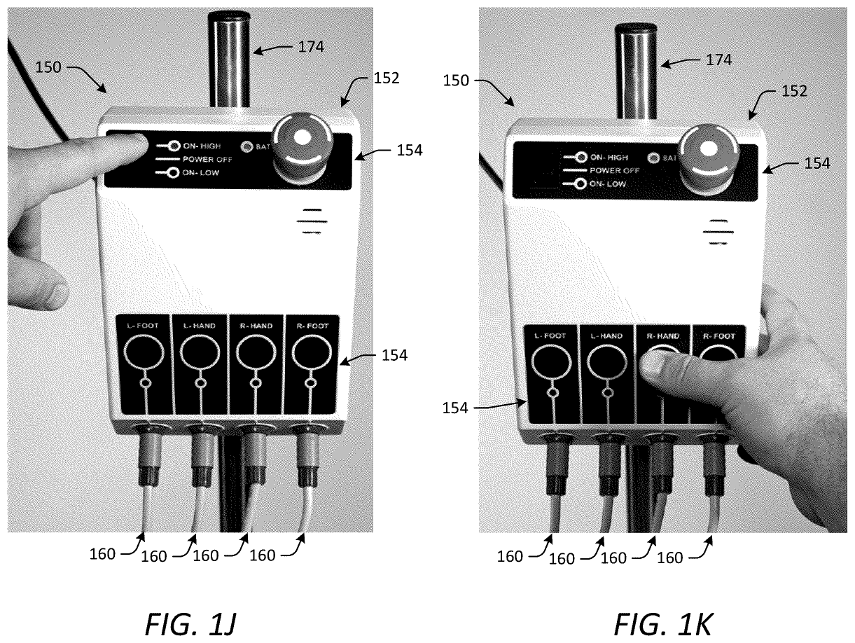

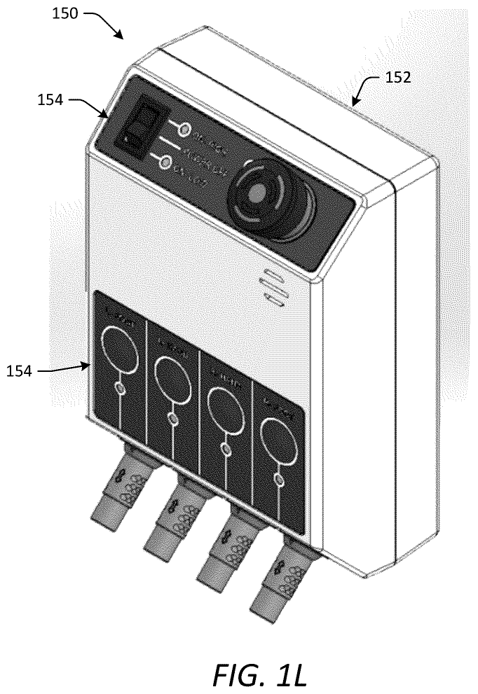

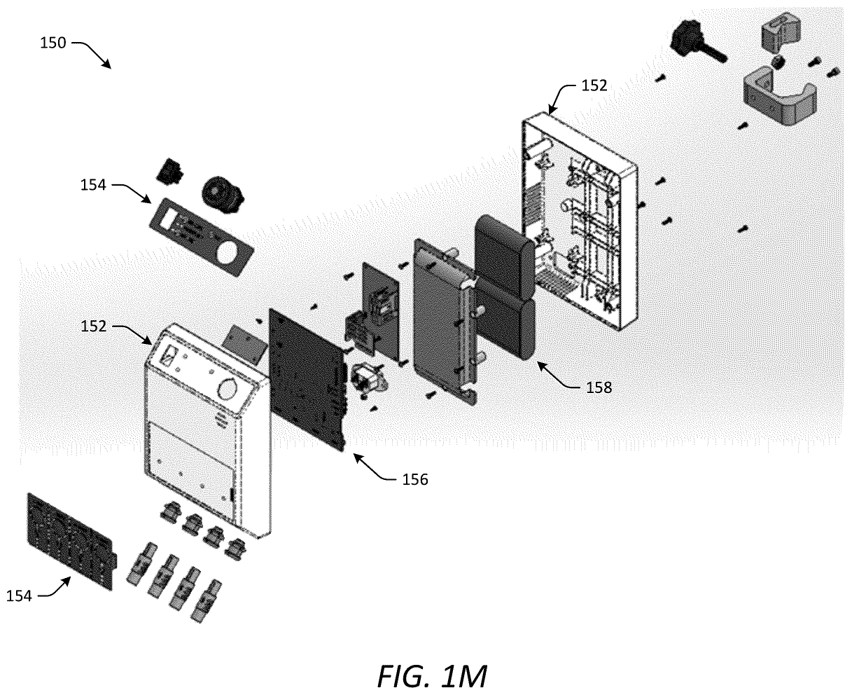

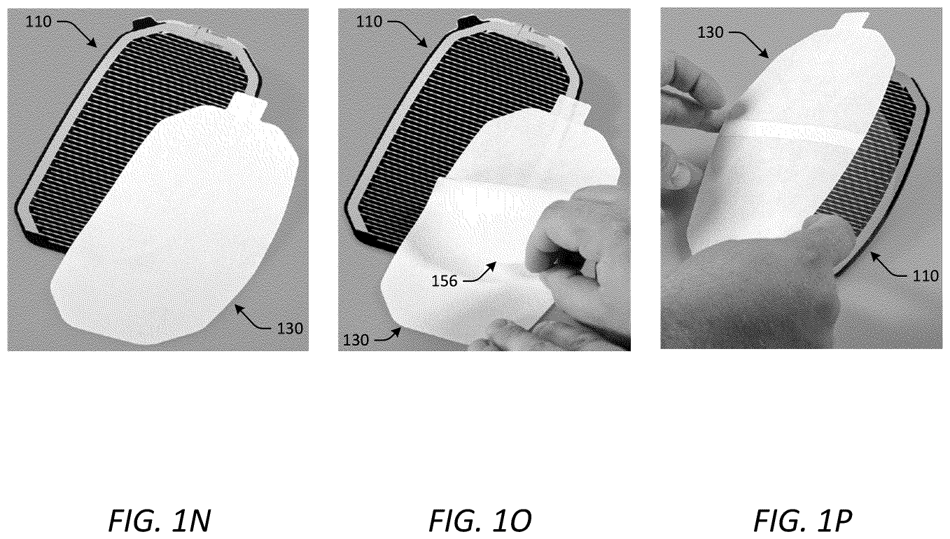

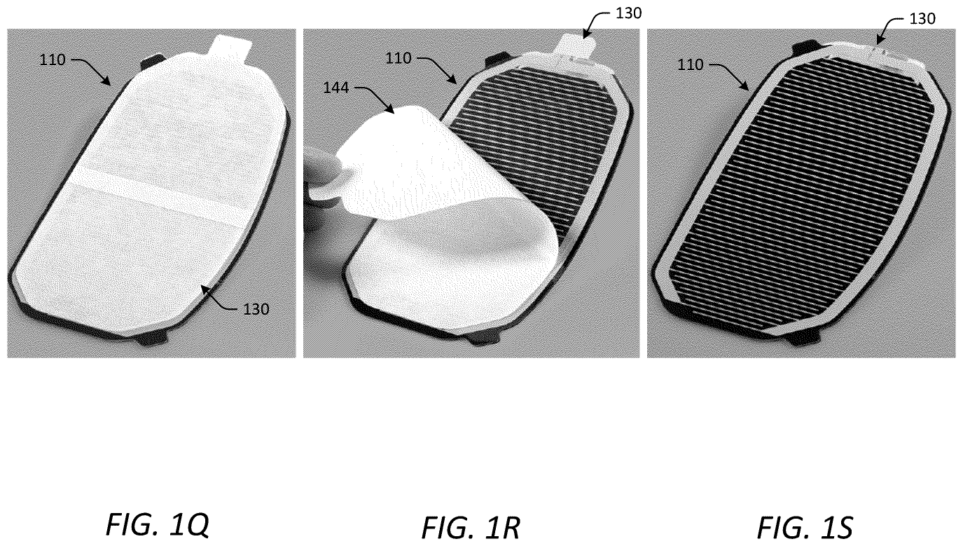

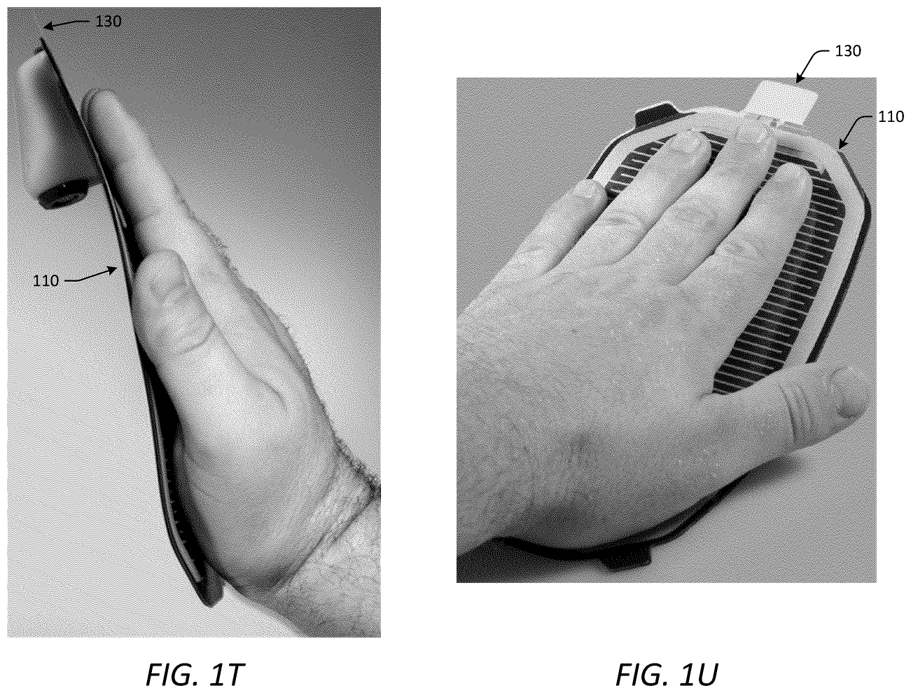

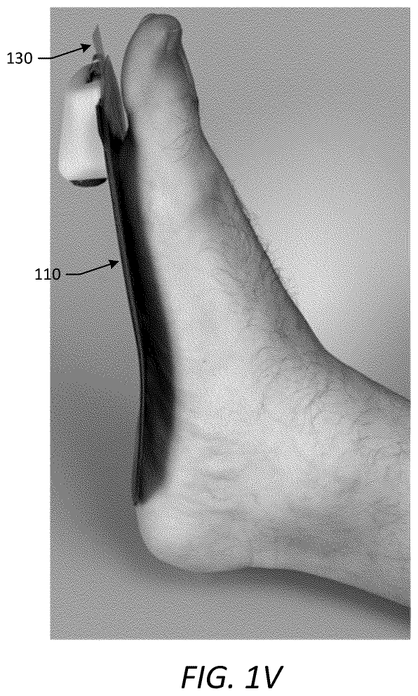

Referring now to FIG. 1A, an example temperature regulation system 100 (which also may be referred to simply as a “system”) for regulating a core body temperature of a living subject is depicted. FIGS. 1B-1V illustrate respective portions, components, and/or subassemblies of the system 100, as described herein.

As shown, the temperature regulation system 100 may include a plurality of flexible heat exchange pads 110, a plurality of flexible adhesive composite laminates 130, a control module 150, a plurality of cables 160, and a support structure 170. According to the illustrated example, four (4) of the flexible heat exchange pads 110 and four (4) of the cables 160 may be provided, such that one of the flexible heat exchange pads 110 may be coupled to each of a subject's hands and feet, with each of the four channels being independently controlled by the control module 150. Fewer or more of the flexible heat exchange pads 110 and the cables 160 may be used in other embodiments. In some embodiments, the flexible heat exchange pads 110, the control module 150, the cables 160, and the support structure 170 may be reusable, while the flexible adhesive composite laminates 130 may be single-use, disposable portions of the system 100.

FIGS. 1B-1E illustrate one of the flexible heat exchange pads 110, each of which may be configured in the same manner. As shown, the flexible heat exchange pad 110 may have a first surface 112 (which also may be referred to as an “active surface”) and a second surface 114 (which also may be referred to as an “inactive side”) disposed opposite one another. The first surface 112 of the flexible heat exchange pad 110 may be configured for facing toward the skin surface when the pad 110 is coupled to the skin surface via one of the flexible adhesive composite laminates 130, and the second surface 114 of the pad 110 may be configured for facing away from the skin surface when the pad 110 is coupled to the skin surface via the flexible adhesive composite laminate 130. As shown, the flexible heat exchange pad 110 may include a flexible heater 116, a flexible thermally-insulative layer 122, a plurality of feedback control temperature sensors 124 disposed on a sensor strip 126, and an electronics module 128. The flexible heater 116 may define the first surface 112 of the flexible heat exchange pad 110 and may be configured for generating heat. In some embodiments, the flexible heater 116 may be a positive temperature coefficient (PTC) heater. As shown, the flexible heater 116 may include a bus bar 118 extending along a periphery of the heater 116, and a plurality of heating elements 120 extending inward from the bus bar 118. In some embodiments, as shown, each of the heating elements 120 may have a linear shape extending in a linear manner from the bus bar 118. As shown, the heating elements 120 may extend parallel to one another and may be arranged in an array. The flexible thermally-insulative layer 122 may define the second surface 114 of the flexible heat exchange pad 110 and may be configured for inhibiting heat transfer from the second surface 114 of the pad 110. As shown, the feedback control temperature sensors 124 may be disposed between the flexible heater 116 and the flexible thermally-insulative layer 122, with the feedback control temperature sensors 124 being coupled to the sensor strip 126. In some embodiments, each of the feedback control temperature sensors 124 may include a resistance temperature detector (RTD). The electronics module 128 may be in operable communication with the flexible heater 116 and the feedback control temperature sensors 124. As shown, the electronics module 128 may be configured for removably coupling with one of the cables 160 via mating connectors.

FIGS. 1F and 1G illustrate an example configuration of the flexible adhesive composite laminate 130 configured for use with the flexible heat exchange pads 110. As shown, the flexible adhesive composite laminate 130 may include a flexible substrate 132, a first adhesive 134, a second adhesive 136, a first cover 144, and a second cover 146. The flexible substrate 132 may have a first surface and a second surface disposed opposite one another, with the first adhesive 134 disposed on the first surface of the flexible substrate 132 and configured for removably adhering to the skin surface, and with the second adhesive 136 being disposed on the second surface of the flexible substrate 132 and configured for removably adhering to the flexible heat exchange pad 110. The second adhesive 136 may be different from the first adhesive 134. The first cover 144 may removably cover the first adhesive 134 and the first surface of the flexible substrate 132 and may be configured for being removed therefrom prior to adhering the first adhesive 134 to the skin surface. The second cover may removably cover the second adhesive 136 and the second surface of the flexible substrate 132 and may be configured for being removed therefrom prior to adhering the second adhesive 136 to the flexible heat exchange pad 110. FIGS. 1N-1S sequentially illustrate removal of the covers 144, 146 and adhering the flexible adhesive composite laminate 130 to the flexible heat exchange pad 110 via the second adhesive 136 in preparation for adhering the flexible adhesive composite laminate 130, together with the flexible heat exchange pad 110, to the skin surface via the first adhesive 134. FIGS. 1T and 1U show an example of the flexible heat exchange pad 110 coupled to a subject's hand via the flexible adhesive composite laminate 130. FIG. 1V shows the flexible heat exchange pad 110 coupled to a subject's foot via the flexible adhesive composite laminate 130.

In some embodiments, the first adhesive 134 may cover at least a majority of the first surface of the flexible substrate 132, and the second adhesive 136 may cover at least a majority of the second surface of the flexible substrate 132. In some embodiments, the first adhesive 134 may cover an entirety of the first surface of the flexible substrate 132, and the second adhesive 136 may cover an entirety of the second surface of the flexible substrate 132. In some embodiments, the flexible substrate 132 may be a film. In some embodiments, the flexible substrate 132 may include a polymer. In some embodiments, the flexible substrate 132 may include polyester. In some embodiments, the first adhesive 134 may be biocompatible. In some embodiments, the second adhesive 136 may be biocompatible. In some embodiments, the second adhesive 136 may be non-biocompatible. In some embodiments, the first adhesive 134 may be a pressure sensitive adhesive. In some embodiments, the first adhesive 134 may be an acrylic adhesive. In some embodiments, the first adhesive 134 may include rubber. In some embodiments, the second adhesive 136 may be a high tack adhesive. In some embodiments, the flexible heat exchange pad 110 may have a first footprint (i.e., overall surface area), and the flexible adhesive composite laminate 130 may have a second footprint that is equal to or less than the first footprint.

FIGS. 1H-1M illustrate an example configuration of the control module 150 configured for use with the flexible heat exchange pads 110. As shown, the control module 150 may include a housing 152, a user interface 154, a controller 156, and a battery 158. The control module 150 may be in operable communication with each of the flexible heat exchange pads 110 via respective cables 160. As shown, each of the cables 160 may be removably coupled to the control module 150 via mating connectors. The user interface 154 may be configured for allowing a user of the system 100 to selectively and independently control operation of the different flexible heat exchange pads 110, as desired. The controller 156 may be configured for controlling operation of the flexible heat exchange pads 110 in accordance with user inputs received via the user interface 154 as well as signals received from the feedback control temperature sensors 124 of the respective pads 110. The battery 158 may be configured for powering the control module 150 and the flexible heat exchange pads 110 when mains electrical power is not available, for example, when the system 100 is being used in a mobile manner during transport of the subject.

As shown in FIG. 1A, the support structure 170 may include a mobile base 172, a support pole 174, and a receptacle 176. The mobile base 172 may allow for ease of transport of the system 100 along the ground. The support pole 174 may extend upward from the mobile base 172 and support the control module 150 and the receptacle 176 thereon. As shown, the receptacle 176 may be configured for storing the flexible heat exchange pads 110 and the cables 160 when not in use.

It will be appreciated that the arrangement and configuration of the different portions, components, and subassemblies of the temperature regulation system 100 illustrated in FIGS. 1A-1V are merely examples, and that various other arrangements and configurations of the system may be used in other embodiments.

### Example Experimental Data

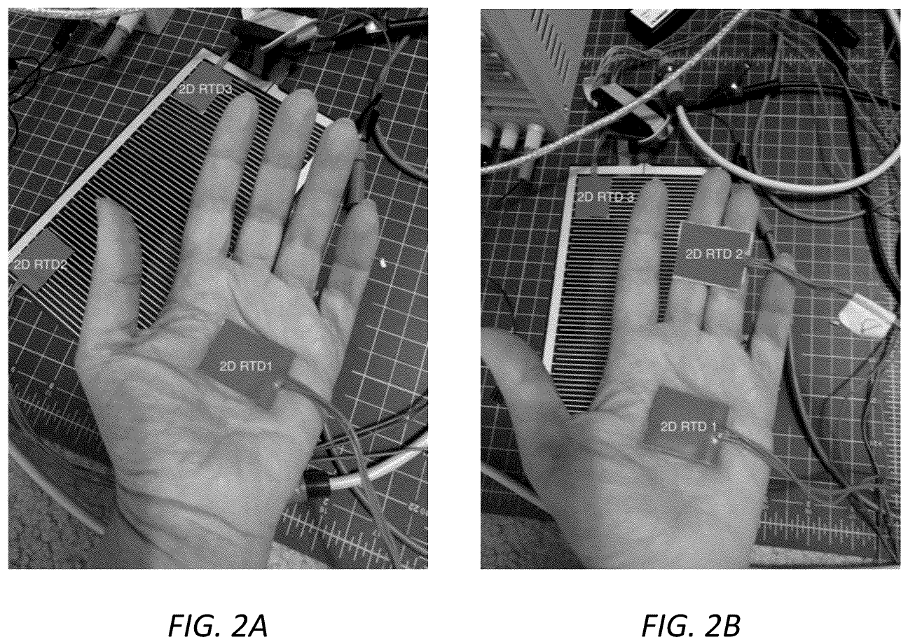

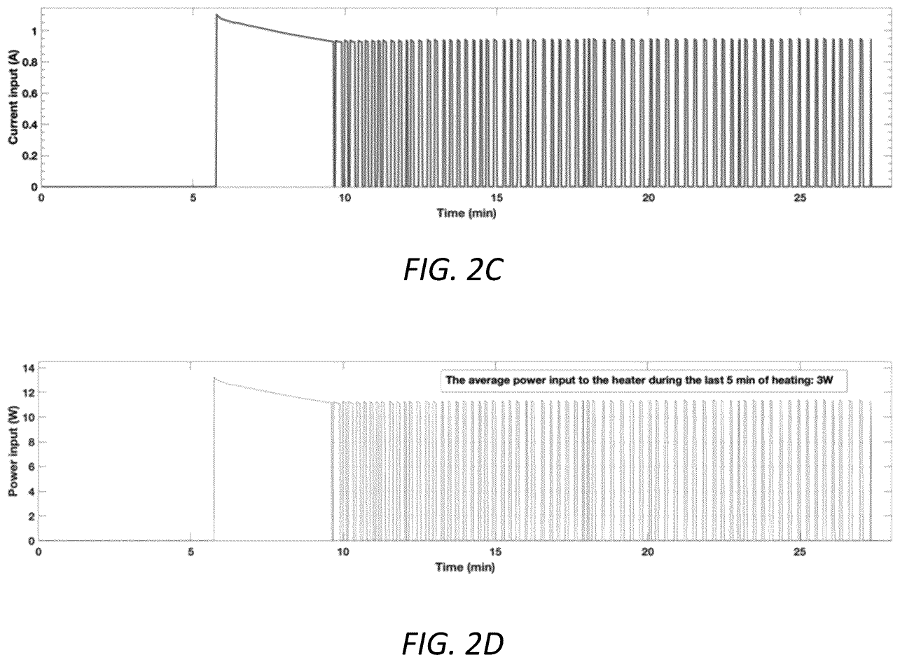

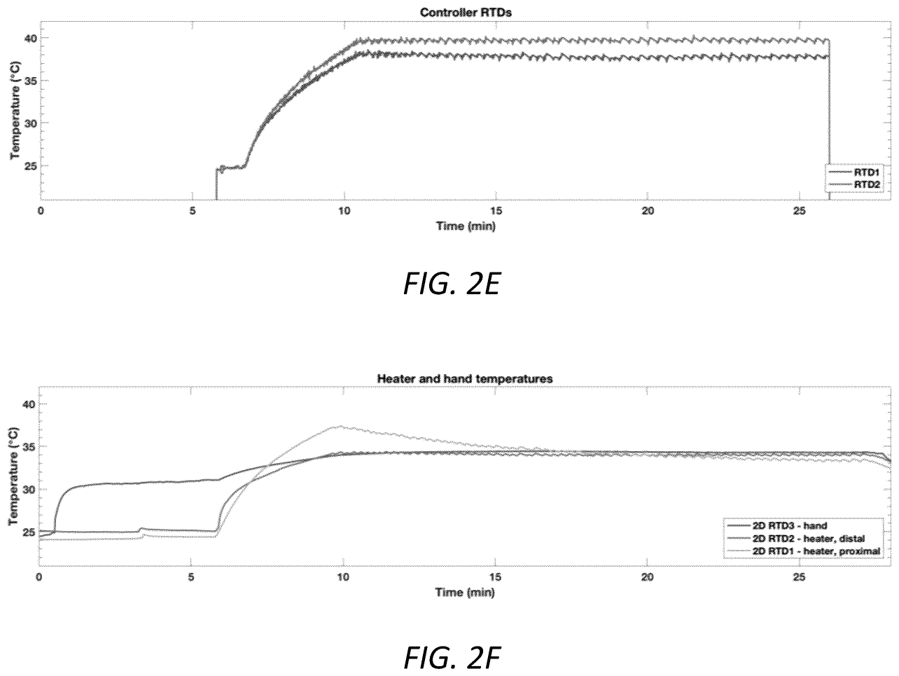

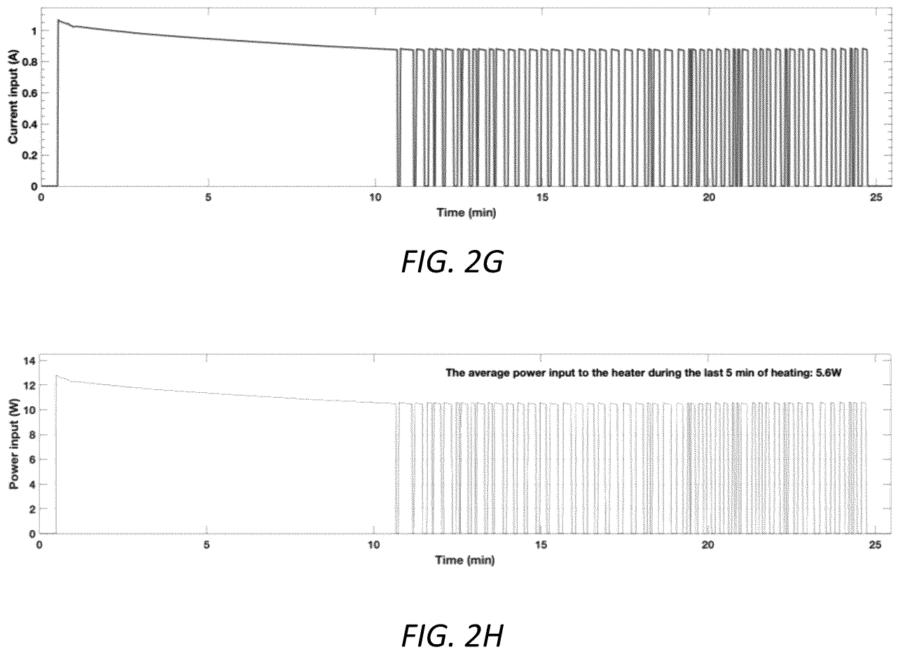

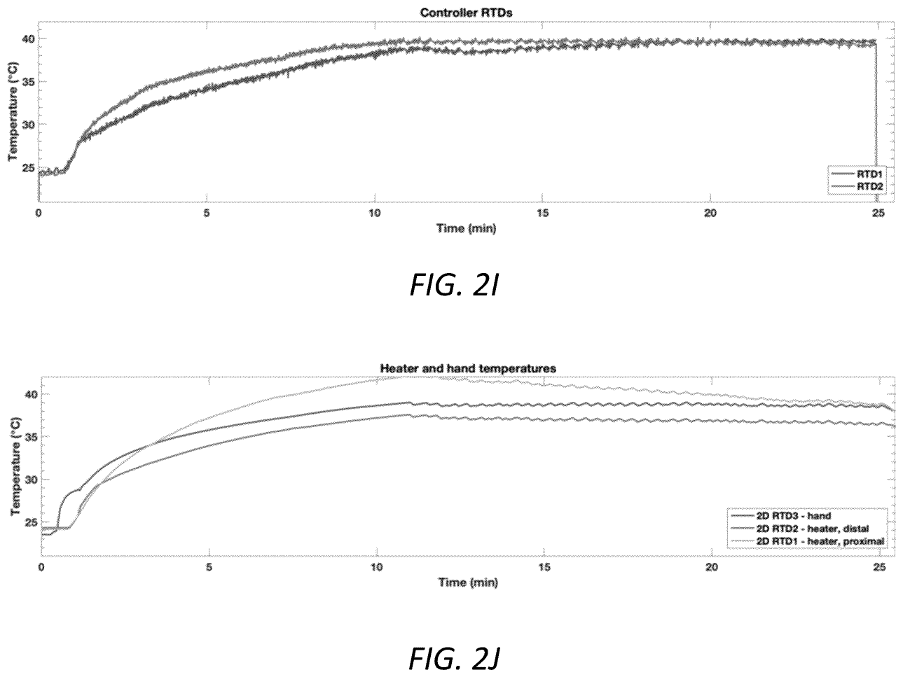

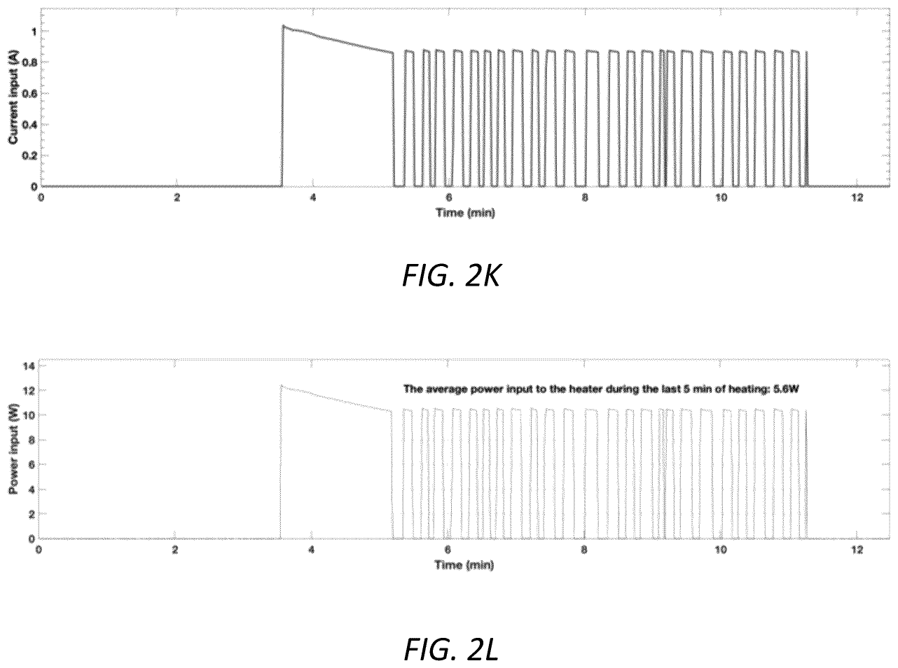

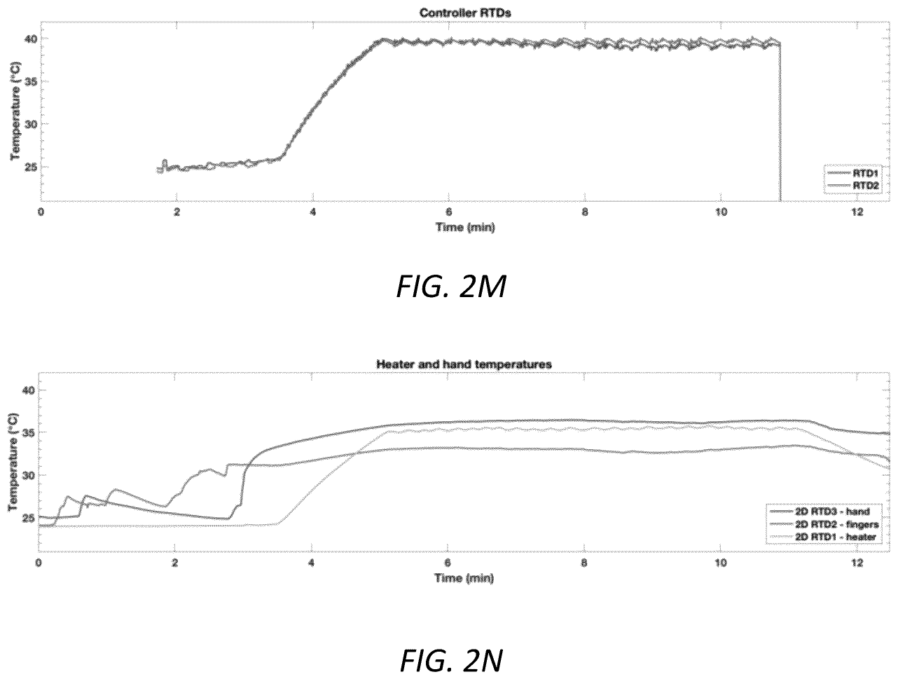

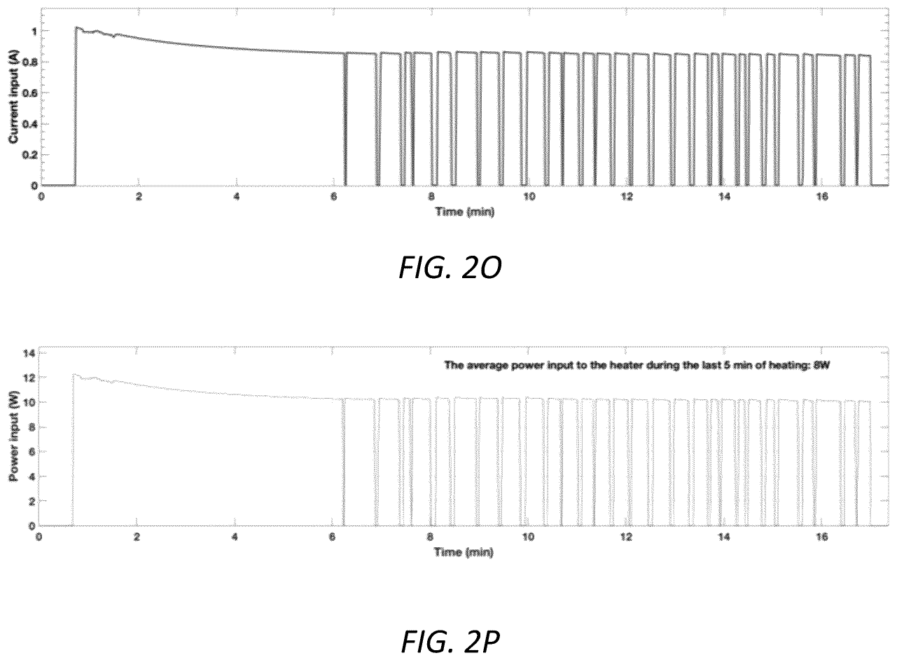

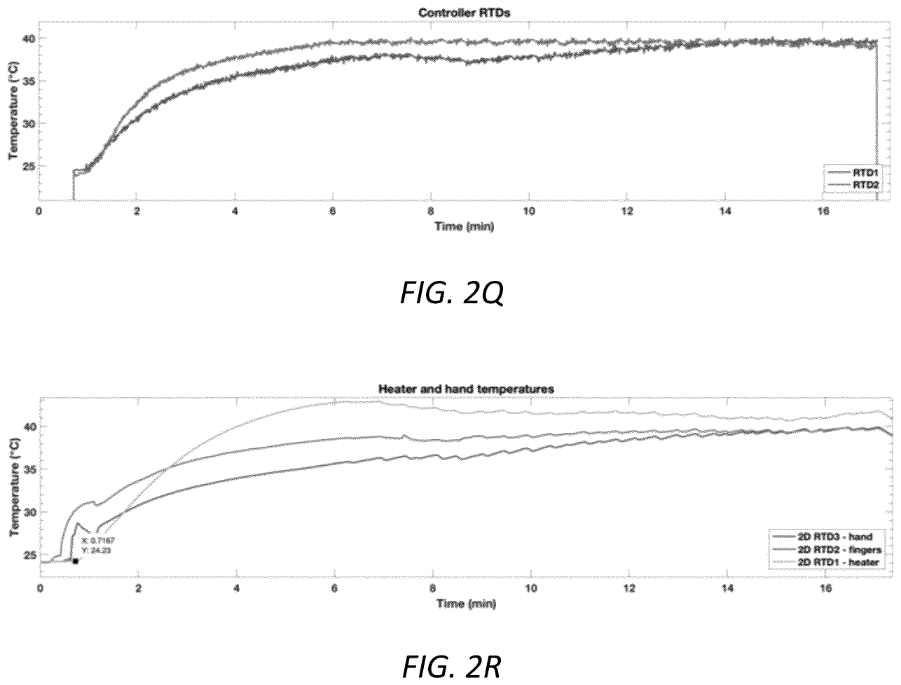

FIGS. 2A and 2B illustrate an experimental test setup that was used for studying the effect of the flexible adhesive composite laminate 130 being used to couple the flexible heat exchange pad 110 to a hand of a human subject. In particular, the testing was conducted to compare hand temperature with and without use of the flexible adhesive composite laminate 130 and to compare power input to the heater 116 between these two conditions.

According to a first test run, the subject's hand was placed face down on the flexible heat exchange pad 110, without the flexible adhesive composite laminate 130 being used. FIGS. 2C-2F show example data obtained from the first test run. FIG. 2C illustrates current input as a function of time. FIG. 2D illustrates power input as a function of time. FIG. 2E illustrates feedback temperature as a function of time. FIG. 2F illustrates hand and heater temperatures as a function of time.

According to a second test run, the subject's hand was placed face down on the flexible heat exchange pad 110, with the flexible adhesive composite laminate 130 being used to couple the pad 110 to the hand. FIGS. 2G-2J show example data obtained from the first test run. FIG. 2G illustrates current input as a function of time. FIG. 2H illustrates power input as a function of time. FIG. 2I illustrates feedback temperature as a function of time. FIG. 2J illustrates hand and heater temperatures as a function of time.

According to a third test run, the subject's hand was placed face up on the flexible heat exchange pad 110, without the flexible adhesive composite laminate 130 being used. FIGS. 2K-2N show example data obtained from the first test run. FIG. 2K illustrates current input as a function of time. FIG. 2L illustrates power input as a function of time. FIG. 2M illustrates feedback temperature as a function of time. FIG. 2N illustrates hand and heater temperatures as a function of time.

According to a second test run, the subject's hand was placed face up on the flexible heat exchange pad 110, with the flexible adhesive composite laminate 130 being used to couple the pad 110 to the hand. FIGS. 2O-2R show example data obtained from the first test run. FIG. 2O illustrates current input as a function of time. FIG. 2P illustrates power input as a function of time. FIG. 2Q illustrates feedback temperature as a function of time. FIG. 2R illustrates hand and heater temperatures as a function of time.

Although specific embodiments of the disclosure have been described, one of ordinary skill in the art will recognize that numerous other modifications and alternative embodiments are within the scope of the disclosure. For example, while various illustrative implementations and structures have been described in accordance with embodiments of the disclosure, one of ordinary skill in the art will appreciate that numerous other modifications to the illustrative implementations and structures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

1. A method for regulating a core body temperature of a living subject, the method comprising:
removably coupling a flexible heat exchange pad to a skin surface of the living subject via a flexible adhesive composite laminate; and
applying heat via the flexible heat exchange pad to the skin surface and underlying tissue of the living subject while the flexible heat exchange pad is coupled to the skin surface via the flexible adhesive composite laminate such that the applied heat regulates the core body temperature of the living subject.

2-3. (canceled)

4. The method of claim 1, wherein the skin surface comprises at least a portion of a hand of the living subject.

5-12. (canceled)

13. The method of claim 1, wherein the flexible heat exchange pad is coupled to the living subject solely by the flexible adhesive composite laminate such that no mechanical pressure is applied to the skin surface or the underlying tissue through the flexible heat exchange pad.

14-19. (canceled)

20. The method of claim 1, wherein removably coupling the flexible heat exchange pad to the skin surface of the living subject via the flexible adhesive composite laminate comprises conforming the flexible adhesive composite laminate and the flexible heat exchange pad to a contour of the skin surface.

21-22. (canceled)

23. The method of claim 1, wherein the living subject is under general anesthesia.

24-42. (canceled)

43. The method of claim 1, wherein the flexible heat exchange pad has a first surface and a second surface disposed opposite one another, wherein the first surface of the flexible heat exchange pad faces toward the skin surface when the flexible heat exchange pad is coupled to the skin surface via the flexible adhesive composite laminate, and wherein the second surface of the flexible heat exchange pad faces away from the skin surface when the flexible heat exchange pad is coupled to the skin surface via the flexible adhesive composite laminate, wherein the flexible heat exchange pad comprises:
a flexible heater defining the first surface of the flexible heat exchange pad and configured for generating heat; and
a flexible thermally-insulative layer defining the second surface of the flexible heat exchange pad and configured for inhibiting heat transfer from the second surface of the flexible heat exchange pad.

44-52. (canceled)

53. The method of claim 43, wherein the flexible heat exchange pad further comprises a plurality of feedback control temperature sensors disposed between the flexible heater and the flexible thermally-insulative layer.

54-56. (canceled)

57. The method of claim 53, further comprising:
determining, via the feedback control temperature sensors, a plurality of temperature values; and
controlling, via an electronic controller, the heat applied via the flexible heat exchange pad to the skin surface and the underlying tissue of the living subject based at least in part on the plurality of temperature values.

58. The method of claim 57, wherein determining, via the feedback control temperature sensors, the plurality of temperature values comprises:
determining, via a first feedback control temperature sensor, a first temperature value; and
determining, via a second feedback control temperature sensor, a second temperature value.

59. The method of claim 58, wherein controlling, via the electronic controller, the heat applied via the flexible heat exchange pad to the skin surface and the underlying tissue of the living subject based at least in part on the plurality of temperature values comprises:
determining a greater temperature value of the first temperature value and the second temperature value;
comparing the greater temperature value and a predetermined maximum temperature setting; and
controlling, via the electronic controller, the heat applied via the flexible heat exchange pad to the skin surface and the underlying tissue of the living subject based at least in part on a comparison of the greater temperature value and the predetermined maximum temperature setting.

60. The method of claim 58, wherein controlling, via the electronic controller, the heat applied via the flexible heat exchange pad to the skin surface and the underlying tissue of the living subject based at least in part on the plurality of temperature values comprises:
determining a differential between the first temperature value and the second temperature value;
comparing the differential and a predetermined threshold limit; and
controlling, via the electronic controller, the heat applied via the flexible heat exchange pad to the skin surface and the underlying tissue of the living subject based at least in part on a comparison of the differential and the predetermined threshold limit.

61. A flexible heat exchange pad for regulating a core body temperature of a living subject, the flexible heat exchange pad comprising:
a flexible heater defining a first surface of the flexible heat exchange pad and configured for generating heat, wherein the first surface of the flexible heat exchange pad is configured for facing toward a skin surface of the living subject when the flexible heat exchange pad is coupled to the skin surface, and wherein the flexible heater has constitutive properties such that the flexible heater is self-regulating against heating above a predetermined temperature; and
a flexible thermally-insulative layer defining an opposite second surface of the flexible heat exchange pad and configured for inhibiting heat transfer from the second surface of the flexible heat exchange pad, wherein the second surface of the flexible heat exchange pad is configured for facing away from the skin surface when the flexible heat exchange pad is coupled to the skin surface.

62. (canceled)

63. The flexible heat exchange pad of claim 61, wherein the predetermined temperature is less than or equal to 43° C.

64-69. (canceled)

70. The flexible heat exchange pad of claim 61, wherein the flexible heat exchange pad further comprises a plurality of feedback control temperature sensors each comprising a resistance temperature detector.

71-90. (canceled)

91. A heat exchange assembly for regulating a core body temperature of a living subject, the heat exchange assembly comprising:
a flexible heat exchange pad comprising:
a flexible heater defining a first surface of the flexible heat exchange pad and configured for generating heat, wherein the first surface of the flexible heat exchange pad is configured for facing toward a skin surface of the living subject when the flexible heat exchange pad is coupled to the skin surface, and wherein the flexible heater has constitutive properties such that the flexible heater is self-regulating against heating above a predetermined temperature; and
a flexible thermally-insulative layer defining an opposite second surface of the flexible heat exchange pad and configured for inhibiting heat transfer from the second surface of the flexible heat exchange pad, wherein the second surface of the flexible heat exchange pad is configured for facing away from when the flexible heat exchange pad is coupled to the skin surface; and
a flexible adhesive composite laminate configured for removably coupling the flexible heat exchange pad to the skin surface, the flexible adhesive composite laminate comprising:
a flexible substrate having a first surface and a second surface disposed opposite one another;
a first adhesive disposed on the first surface of the flexible substrate and configured for removably adhering to the skin surface; and
a second adhesive disposed on the second surface of the flexible substrate and configured for removably adhering to the flexible heat exchange pad, wherein the second adhesive is different from the first adhesive.

92-94. (canceled)

95. The heat exchange assembly of claim 91, wherein the flexible heater comprises:
a bus bar extending along a periphery of the flexible heater; and
a plurality of heating elements extending inward from the bus bar.

96-99. (canceled)

100. The heat exchange assembly of claim 91, wherein the flexible heat exchange pad further comprises a plurality of feedback control temperature sensors.

101. (canceled)

102. The heat exchange assembly of claim 100, wherein the feedback control temperature sensors are coupled to a sensor strip, and wherein the sensor strip is disposed between the flexible heater and the flexible thermally-insulative layer.

103-117. (canceled)

118. The heat exchange assembly of claim 91, wherein the flexible adhesive composite laminate further comprises:
a first cover removably covering the first adhesive and the first surface of the flexible substrate and configured for being removed therefrom prior to adhering the first adhesive to the skin surface; and
a second cover removably covering the second adhesive and the second surface of the flexible substrate and configured for being removed therefrom prior to adhering the second adhesive to the flexible heat exchange pad.

119. (canceled)

120. The heat exchange assembly of claim 91, wherein the flexible heat exchange pad has a first footprint, and wherein the flexible adhesive composite laminate has a second footprint that is equal to or less than the first footprint.
